(19) United States
(12) Patent Application Publication
(10) Pub. No.: US 2017/0266429 A1
(43) Pub. Date: Sep. 21, 2017

(54) Connecting system, injection system for the injection of a medical fluid and method of operating such an injection system

(71) Applicant: ulrich GmbH & Co. KG, Ulm (DE)

(72) Inventors: Uwe STRIGGOW, Aichtal (DE); Felix SEIBOLD, Ulm (DE); Markus GÖTZ, Blaustein (DE)

(73) Assignee: ulrich GmbH & Co. KG, Ulm (DE)

(21) Appl. No.: 15/458,119

(22) Filed: Mar. 14, 2017

(30) Foreign Application Priority Data
Mar. 17, 2016 (DE) 10 2016 104 937.9

(51) Int. Cl. A61M 39/10; A61M 5/142; A61M 5/14

(52) U.S. Cl. CPC A61M 39/10; A61M 5/1413; A61M 5/142; A61M 2205/6018; A61M 2205/12; A61M 2205/502; A61M 2205/18; A61M 2205/11

(57) ABSTRACT

A connecting system for connecting a medical tubular line to a medical fluid transfer system, the tubular line including a plug-in component with a first fluid channel and the fluid transfer system including a coupling component with a second fluid channel. The plug-in component can engage in the coupling component to establish a passageway for a medical fluid, which passageway connects the fluid channels to one another. By disposing a first passive transponder in the plug-in component and a second passive transponder in the coupling component at a distance below a predefined threshold value relative to one another, communication of a read/write unit with both transponders via a single antenna is made possible when the plug-in component is engaged in the coupling component so that identification data for the identification of the tubular line and the fluid transfer system stored in the transponders can be read by a read/write unit via a single antenna.

### FIELD OF THE INVENTION

The present invention relates to a connecting system for connecting a medical tubular line to a medical fluid transfer system as well as to an injection system for the injection of a medical fluid and to a method of operating such an injection system.

### BACKGROUND

The intravenous injection of medical fluids into the body of humans is an intervention fraught with risk which requires the utmost care and the strict compliance with hygiene rules. To prevent medical errors in this area, it has already been proposed to use RFID transponders. Thus, EP 2 170 428 A1 describes the use of RFID transponders in a dialysis machine. During dialysis, a mixture of a plurality of different fluids is injected into the venous blood stream of a patient, with mixing in the dialysis machine taking place only immediately prior to the injection. To avoid mix-ups, it is therefore proposed that in the prior-art dialysis machine, RFID transponders which can be read by a read unit of the dialysis machine be disposed on tubular lines that are connected to the fluid containers and on a cassette which can be replaceably inserted in the dialysis machine, in which cassette the various fluids from the fluid containers are mixed. To implement this, a plurality of antennas of the read unit are integrated into an automatic connecting mechanism which, controlled by a microcomputer, respectively connects and disconnects the tubular lines of the fluid containers to and from the cassette.

### SUMMARY

The disclosure relates to a connecting system for connecting a medical tubular line to a medical fluid transfer system as well as an injection system which, without requiring complex equipment, allows the medically correct use of the connected components to be monitored, and to specify a suitable method of operating an injection system while using such a connecting system.

Accordingly, a connecting system, an injection system for the injection of a medical fluid, and a related method are disclosed. Preferred embodiments are also disclosed.

The connecting system, such as is disclosed by the present invention, for connecting a medical tubular line to a medical fluid transfer system comprises a plug-in component with a first fluid channel and a coupling component with a second fluid channel, with the plug-in component being disposed on the tubular line and the coupling component being disposed on the fluid transfer system, and with the plug-in component being able to engage in the coupling component so as to create a passageway for a medical fluid, which connects the fluid channels to one another. According to the present invention, a first passive transponder is integrated into the plug-in component, and a second passive transponder is integrated into the fluid transfer system, with the spacing between the first transponder and the second transponder being below a predefined threshold limit which is selected to ensure that communication of a read/write unit with both transponders is possible via a single antenna when the plug-in component is engaged in the coupling component.

The first transponder makes it possible to monitor the connection of an appropriate and correct tubular line to the fluid transfer system directly on the connecting system independently of the path of the tubular line at a distance from the plug-in component on which the tubular line ends. When the plug-in component is connected to the coupling component of the fluid transfer system, it occupies a defined position relative to the fluid transfer system, and the first transponder that is integrated in the plug-in component can be read from and/or written to by a read/write unit, the antenna of which is appropriately positioned on an injection system with respect to the coupling component of the fluid transfer system. A guide component for the tubular line, which ensures appropriate positioning of a transponder disposed on the tubular line with respect to an antenna of a read/write unit, is therefore not required.

According to the present invention, a second passive transponder is integrated in the coupling component, which makes it possible to additionally monitor that an appropriate and correct fluid transfer system is used. To enable both transponders to be optionally read from or written to simultaneously by a read/write unit, the spacing between the first transponder integrated in the plug-in component and the second transponder disposed on the coupling component below a predefined threshold limit is so small that the read/write unit is able to communicate with both transponders via a single antenna when the plug-in component is engaged in the coupling component. It is then possible to use a read/write unit with a single antenna.

It is recommended that the first transponder and the second transponder be configured in the form of RFID transponders and that each transponder have a first read-only memory (ROM) containing identification data that are unchangeably stored therein. In addition thereto, each transponder can also have a second readable as well as writable memory. The read-only memory contains, for example, an identification code, based on which an appropriate original component can be distinguished from an unauthorized or inappropriate component. The optionally additionally provided writable memory allows data to be stored while the respective component (tubular line or fluid transfer system) is being used.

Equipping a plug-in component of a medical tubular line with a transponder, such as is disclosed by the present invention, is especially useful if the tubular line, on its end facing away from the plug-in component, has a connector that is configured for connecting a cannula, i.e., that is designed to lead directly to a patient who is to receive an intravenous injection of fluid, since in this case especially strict hygiene requirements prevail, which can be automated and securely monitored and safeguarded by the electronic monitoring system. The same applies to a fluid transfer system if this system is a cassette which has a plurality of input ports for the delivery of medical fluids and a plurality of fluid channels and valves and which can be inserted into an injection system for the intravenous injection of a medical fluid into the body of humans or animals, with the output port of said cassette being disposed on the coupling component.

The connecting system according to the present invention can be used, for example, in an injection system for the intravenous injection of a medical fluid. Such an injection system comprises a housing, a delivery device for the delivery of the fluid and a fluid transfer system which can be connected to at least one fluid container and can be exchangeably inserted in the housing and which comprises the coupling component and a tubular line that is connected to the coupling component via the plug-in component, with the plug-in component being coupled to the coupling component so as to establish a connection. According to the present invention, a first passive transponder is integrated in the plug-in component and/or a second passive transponder is integrated in the coupling component.

It is recommended that a read/write unit for reading from and/or writing to the first and/or the second transponder be disposed on the housing of the injection system, with the read/write unit requiring, as a result of the arrangement of the two transponders close to one another, only a single antenna for reading from or writing to both the first transponder and the second transponder when the fluid transfer system is coupled to the tubular line via the connecting system. Thus, the transponders disposed in the plug-in component and in the fluid transfer system can optionally be read from or written to simultaneously. The read/write unit preferably has only a single antenna, by means of which it can communicate with the two transponders.

A method, such as is disclosed by the present invention, of operating such an injection system for the intravenous injection of a medical fluid into the body of humans or animals comprises the following steps:
a. Inserting the fluid transfer system into the housing of the injection system and connecting the fluid transfer system to at least one fluid container,
b. Establishing a connection between the fluid transfer system and the tubular line via the connecting system,
c. Reading the identification data for the fluid transfer system (2) that are stored in the first transponder and the identification data for the tubular line that are stored in the second transponder by means of the read/write unit.

Using this method, it is possible to read, and compare, data from the first transponder and from the second transponder prior to starting the delivery of a fluid that is to be injected, with the delivery starting only if the data read are within a predefined range of admissible values or conform to reference data. This makes it possible, for example, to reliably prevent an injection of a medical fluid into the body of a patient if a tubular line and/or a fluid transfer system, which are/is designed for this use, does/do not comply with the relevant standards or have/has already been previously used for an injection.

Preferably, the data stored in the first transponder that is integrated in the plug-in component contain an identification code which is stored in a read-only memory of the first transponder and/or the data stored in the second transponder that is disposed in the fluid transfer system contain an identification code which is stored in a read-only memory of the second transponder. Based on such an identification code, a (new and sterile) original component (tubular line or fluid transfer system) approved for use in the injection system can be distinguished from an unapproved component of, for example, dubious sterility or from a component that has already been used, and the injection of a fluid into a patient using the unapproved component can be prevented.

To ensure compliance with the relevant hygiene standards, it is especially advantageous if, based on the data read from the first and/or the second transponder prior to starting the delivery of a fluid to be injected, it is determined whether and, optionally, at what point in time a fluid to be injected has previously been delivered for the first time by means of the tubular line and/or the fluid transfer system, and that delivery at the current point in time is started only if the difference in time between the current point in time and the point in time of the first use read [from the transponder(s)] is below a predefined threshold limit. In this context, it should be noted that the predefined threshold limit of the difference in time for the tubular line can differ from that for the fluid transfer system. Thus, the medical staff that uses an injection system is relieved of having to manually record the time at which the individual components are used, and the risk of a violation of the hygiene standards with respect of the length of time of use as a result of errors during manual recording is eliminated.

In order to automate the recording of the length of time of use as much as possible and to reduce the workload of the medical staff, it is recommended that the point in time at which the tubular line is used for the first time be written to a memory of the first transponder if, based on the data read from the first transponder prior to starting the delivery of a fluid to be injected, it is determined that the tubular line will be used for the first time. Similarly, the point in time at which the fluid transfer system is used for the first time can be written to a memory of the second transponder if, based on the data read from the second transponder prior to starting the delivery of a fluid to be injected, it is determined that the fluid transfer system will be used for the first time. Thus, a new component (the tubular line or the fluid transfer system) is automatically marked with the time it was first used, thereby eliminating the need for the medical staff to enter the relevant information.

It is recommended that the length of time of use to date and/or the allowable remaining length of time of use of the tubular line and/or the fluid transfer system be displayed on a display unit of the injection system. This allows the medical staff to prepare in advance for the need of having to use a new component and to ensure that this component is available as soon as it is needed. In addition, the display unit of the injection system can also display an error message whenever the data read from a transponder are outside the predefined range of allowable values in order to inform the medical staff of the reason that an injection procedure has been blocked.

### BRIEF DESCRIPTION OF THE DRAWINGS

These and other advantages and features or the connecting system according to the present invention follow from the embodiment example described in greater detail below with reference to the appended drawings. The drawings show

FIG. 1 a view of a longitudinal section through the coupling component of a connecting system according to the present invention,

FIG. 2 a view of a longitudinal section through the plug-in component of a connecting system according to the present invention,

FIG. 3 a view of a longitudinal section through a connecting system according to the present invention, with the plug-in component plugged into the coupling component and

FIG. 4 a diagram of a method according to the present invention in the form of a process flowchart.

### DETAILED DESCRIPTION

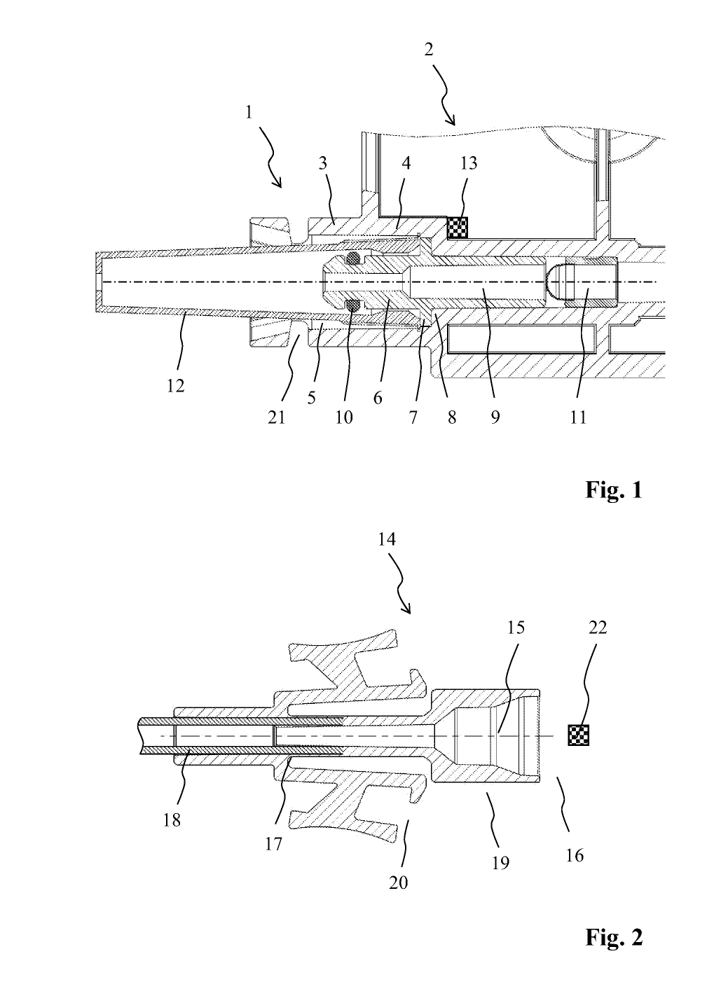

FIG. 1 shows a view of a longitudinal section through the coupling component 1 of a connecting system according to the present invention. This coupling component is an integral part of a cassette 2, which can be replaceably inserted into a housing of an injection system for the injection of contrast agents into the body of humans or animals. On its input side, the cassette 2—only a small portion of which is shown in FIG. 1—comprises a plurality of ports for fluid containers, for example, containers for the contrast agents and a rinsing solution, such as NaCl, as well as fluid channels formed through the housing of the cassette, which channels are covered by a flexible membrane and can be opened and closed from the outside by mechanical actuators that act upon the membrane. Attached to the output side of the cassette 2 is a pump tube bent into the shape of a loop, which pump tube can be inserted into a tube pump of the injection system.

The coupling component 1 constitutes the output port of the cassette 2, the upstream end of which output port is connected to the downstream end of the pump tube and the downstream end of which output port can be connected to a patient tube, which comprises a cannula for puncturing a vein of a patient. Thus, the cassette 2 forms the interface between the containers containing the fluids to be injected and the patient tube, thereby making it possible to avoid direct contact of the injection system with the fluids to be injected.

The coupling component 1 essentially has the shape of a pipe which is disposed on a corner of the housing of the cassette 2 parallel to one of the sides of the cassette. The coupling component 1 is preferably integrally formed in one piece with the cassette housing. An outer section of the coupling component 1 is formed by a projection 3 which protrudes outwardly from the cassette 2. An inner section of the coupling component 1 is formed by a recess 4 which is coaxial with respect to the projection 3 in the housing of the cassette 2. The projection 3 and the recess 4 create a hollow chamber 5 which extends into the cassette 2. In this hollow chamber 5, the coupling component 1 comprises an insert 6 which essentially also has the form of a pipe and which, in the middle section of its length, has a flange 7, by means of which it rests against a shoulder 8 of the recess 4 where it is attached, for example, by means of adhesive bonding. Near the front end of the insert which is overlapped by the projection 3, the insert 6 has an O-ring 10 seated in an annular groove. On its inside, the insert 6 forms a channel 9 for the fluid flowing from the cassette 2 in the direction of the patient tube. On the inside of the cassette 2, a filter 11 is preferably disposed directly upstream of the rearward end of the insert 6, which filter was placed into this area before the insert 6 was installed and is attached to this area by means of adhesive bonding or clamping.

As shown in FIG. 1, when not in use, the coupling component 1 is covered by a cap 12 which is pushed into the hollow chamber 5 up to the flange 7 of the insert 6 and connected to the coupling component 1 by clamping it between the recess 4 and the insert 6. To attach a patient tube to the cassette 2, this connection can be broken by applying a sufficiently high axial tensile force, and the cap 12 can subsequently be removed in an axial direction from the hollow chamber 5.

Disposed on the shoulder 8 of the recess 4 of the coupling component 1 on the inside of the cassette 2 is a passive RFID transponder 13 in which identification data of the cassette 2 are stored. To be able to communicate with the transponder 13, the injection system, in which the cassette 2 is used, contains an RFID read/write unit which is connected to the control unit of the injection system and disposed on a housing of the injection system. The function of the transponder 13 will be discussed in greater detail below. It is preferably undetachably connected to the housing of the cassette 2, for example, cast into or adhesively bonded to the housing.

FIG. 2 shows a view of a longitudinal section through a plug-in component 14 which fits into the coupling component 1. Like the housing of the cassette 2, the plug-in component is a plastic injection molded part, and like the coupling component 1, it has the form of a pipe, with its outside diameter being slightly smaller than the inside diameter of the hollow chamber 5 of the coupling component 1, so that the plug-in component, after removal of the cap 12, can be inserted into the hollow chamber 5 in order to establish a connection to the coupling component 1 so as to create a passageway for a fluid. When not in use, the plug-in component 14 is covered by a cap which is not shown in FIG. 2, the form of which cap is comparable to that of the cap 12 of the coupling component 1. When not in use, this cap encloses the upstream section 16 of the plug-in component 14 from the outside.

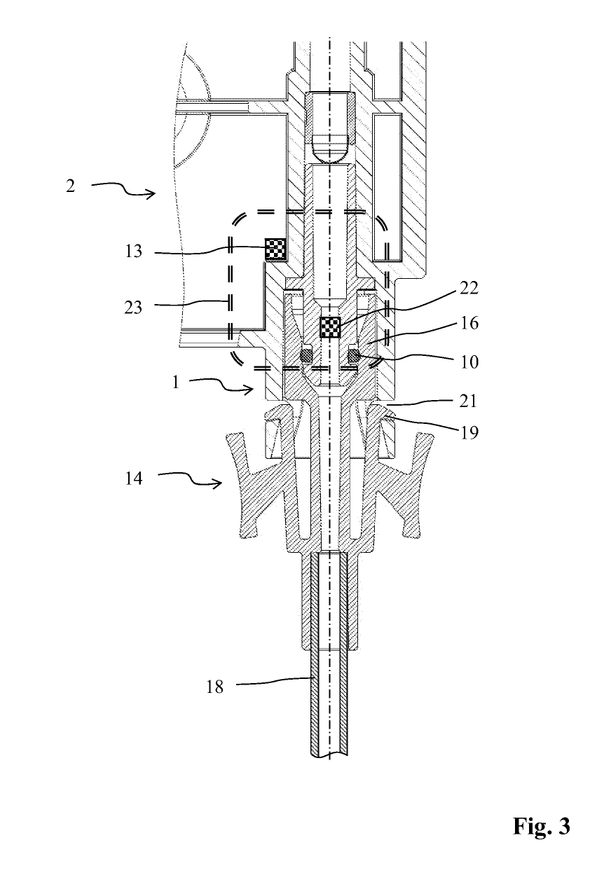

In the upstream section 16 of the plug-in component 14, the channel 15 formed on the inside of the plug-in component 14 widens like a funnel. The upstream section 16 of the plug-in component 14 is designed for being inserted into the coupling component 1. As shown in FIG. 3, when inserted, the section 16 of the plug-in component 14 encloses the downstream end of the insert 6 of the coupling component 1 and seals it with the O-ring 10. This can be seen in the sectional view of FIG. 3 in which the coupling component 1 and the plug-in component 14 are shown to be connected to one another, with the two components being rotated counterclockwise 90° with respect to FIGS. 1 and 2.

The downstream section 17 of the plug-in component 14 which, when connected, faces away from the coupling component 1, forms the connecting port for a tubular line 18 that forms the patient tube, by means of which the output port of the cassette 2 is connected to a patient. One end of the tubular line 18 is firmly connected to the plug-in component 14, for example, by means of adhesive bonding or clamping, and the other end of the tubular line 18 is connected to a cannula (not shown in the drawing).

In in middle section of the plug-in component 14, two diametrically oppositely lying snap-in hooks 19 with grips 20 are integrally formed in one piece with the hooks, which snap-in hooks can be radially inwardly moved by exerting manual pressure on the grips 20. This facilitates inserting the plug-in component 14 into the coupling component 1. To pull the plug-in component 14 out of the coupling component 1, the locked connection between the plug-in component 14 and the coupling component 1 can be disengaged by exerting pressure on the grips 20. As the plug-in component 14 is being connected to the coupling component 1, this locked connection is formed as a result of the interaction between the snap-in hooks 19 and the corresponding cutouts 21 which are provided near the end of the projection 3 in the coupling component 1. As the plug-in component 14 is being inserted into the coupling component 1, the snap-in hooks 19 engage in the diametrically oppositely lying cutouts 21 so as to lock the plug-in component 14 in place on the coupling component 1 after it has been inserted into the hollow chamber 5 as far as it will go in. Because of this locked connection, an unintentional disengagement of the connection is prevented (FIG. 3).

An additional RFID transponder 22 is disposed in the plug-in component 14, more specifically in the upstream section 16 which projects into the hollow chamber 5 inside the coupling component 1 when the connecting system is connected. The transponder 22 is preferably undetachably connected to the plug-in component 14, for example, cast into or adhesively bonded to the plug-in component, and contains identification data for the identification of the tubular line 18 in a memory (ROM). As FIG. 3 shows, when coupled, the RFID transponder 22 of the plug-in component 14 is disposed beyond the O-ring 10 on the insert 6 inside the coupling component 1, and the distance from the RFID transponder 13 of the coupling component 1 is very short. Thus, it is possible to communicate with both RFID transponders 13 and 22 via a single antenna 23 of an RFID read/write unit, the location of which antenna is indicated by the broken line in FIG. 3. As a result, it is possible to simultaneously read the identification data stored in the transponders 22, 22 for the fluid transfer system (cassette 2) and the tubular line 18 connected thereto by means of a read/write unit with a single antenna, which read/write unit is disposed in a housing of an injection system.

The RFID transponders 13 and 22 thereby make it possible to monitor the correct usage of the cassette 2, the integral part of which is the coupling component 1, and of the tubular line 18, the integral part of which is the plug-in component 14. Thus, by reading an identification code from a read-only memory of an RFID transponder 13 or 22, it can be determined whether a cassette 2 or a tubular line 18 is a consumable part that has been authorized by the manufacturer of the injection system and which is suitable for use in the injection system and meets the relevant medical approval requirements.

In addition, a cassette 2 and a tubular line 18 leading to a patient are also subject to hygiene standards that limit the length of time of use, where the allowable length of time of use of a tubular line 18 that leads to a patient is, as a rule, shorter than that of a cassette 2. For example, the allowable length of time of use of a patient tube may be a maximum of 12 hours and that of a cassette may be a maximum of 24 hours. According to the present invention, complying with these standards is ensured in that a timestamp record is read from a memory of an RFID transponder 13 and/or 22, which timestamp record identifies the point in time at which a cassette 2 or a tubular line 18 was first used. A new cassette 2 or a new tubular line 18 will not have such a timestamp record, which means that the memory location dedicated to this purpose in the transponder 13 and 22 still has a content that indicates that the respective component is new, i.e., has not yet been used.

Prior to each start-up operation of the delivery device (tube pump) of the injection system, this timestamp record is read as well, and by comparison with the current date and the current time, it is determined whether the length of time of use to date is still below the maximum allowable value. If this is the case, operation of the delivery device is released, if it is not, a relevant error message will appear on a display unit of the injection system. If a new cassette 2 or a new tubular line 18 is identified, the current point in time is written in the form of a timestamp record to the memory dedicated for this purpose in the transponder 13 or 22 and marks the time of first use.

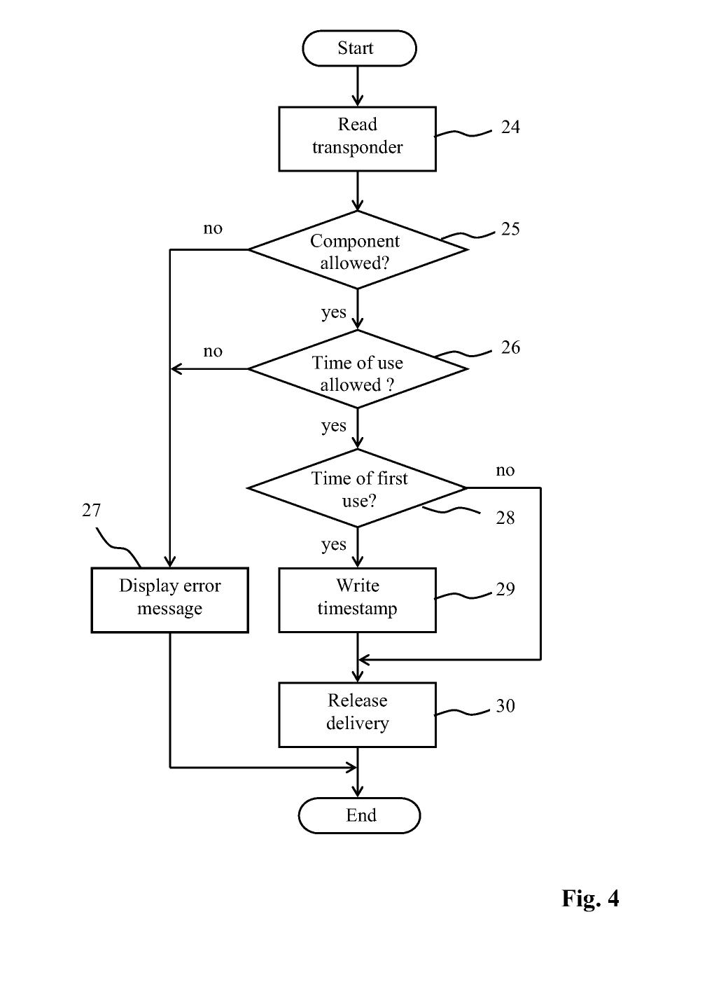

FIG. 4 shows a diagram of this method in the form of a process flowchart, by means of which it is also possible to monitor compliance with the time limits for the use of a cassette 2 and/or a tubular line, such as is disclosed by the present invention. First, in Step 24, the relevant data are read from the transponder 13 and/or 22. In Step 25, it is subsequently checked whether an identification code contained in the data read is correct and identifies the cassette 2 and/or the tubular line 18 as an approved original part. If the answer is yes, it is then further checked in Step 26 whether, based on the timestamp record read and compared with the current time read from a real-time clock of the injection system, the allowable length of time of use is still in compliance. As and when considered advisable, the projected length of time for the injection procedure, prior to the beginning of which the process according to the present invention is concluded, could be factored in, and the allowable length of time of use could be deemed to have been exceeded even if at the time of executing Step 26, it has not yet been exceeded, but would be exceeded within the projected length of time for the upcoming injection, which is known to the control unit of the injection system.

If the result of one of Steps 25 or 26 is negative, an error message is displayed in Step 27, which informs the medical staff about the identified cause of error. In this case, delivery of a fluid through the pump of the injection system is not released and the sequence of the method according to the present invention is concluded.

If the result of Step 26 is positive, an additional check based on the timestamp record read is carried out in Step 28 to determine whether the respective component is used for the first time, i.e., whether the component is new. If the answer is yes, in Step 29, the current point in time is written as the timestamp record to the memory dedicated for this purpose in the transponder 13 and/or 22, before delivery of a fluid through the delivery device of the injection system is released in Step 30. The latter always takes place if the result of both Steps 25 and 26 were positive.

The display unit of the injection system is also used to inform the medical staff about the status of the tubular line 18 and the cassette 2 with respect to time in that it displays the length of time of use to date and/or the allowable remaining length of time of use.

Although not shown in the flowchart of FIG. 4, but useful in the interest of a patient, is the possibility of manually overriding the blocking of the delivery after expiration of the allowable length of time of use of a tubular line 18 or a cassette 2, if this time expires during a series of injections into one and the same patient. In this case, replacing the component involved is not necessary from a medical standpoint, and it would even subject the patient to unnecessary stress if in such a case, the tubular line 18, with the cannula inserted into the body of the patient, were to be replaced. This is the reason for providing the possibility of manually releasing the delivery by entering a relevant command into an input unit of the injection system. Making use of the possibility of manually releasing the delivery in spite of the fact that the allowable length of time of use has expired falls within the responsibility of the medical staff.

The layouts of the coupling component 1 and of the plug-in component 14 shown in FIGS. 1 to 3 are to be understood as examples only. It is apparent to those skilled in the art that a connecting system according to the present invention could also have a different mechanical design, and that the method according to the present invention principally requires only the presence of a transponder on a plug-in component or on a coupling component of a medical connecting system, without requiring said connecting system to have a specific configuration.

What is claimed is:

1. A connecting system for connecting a medical tubular line to a medical fluid transfer system, with the tubular line comprising a plug-in component with a first fluid channel and with the fluid transfer system comprising a coupling component with a second fluid channel, and with the plug-in component being able to engage in the coupling component so as to create a passageway for a medical fluid that connects the fluid channels to one another, wherein a first passive transponder is integrated in the plug-in component, and a second passive transponder is integrated in the coupling component, with the spacing between the first transponder and the second transponder being below a predefined threshold limit which is selected to ensure that communication of a read/write unit with both transponders is possible via a single antenna when the plug-in component is engaged in the coupling component.

2. The connecting system of claim 1, wherein data stored in the first transponder contain identification data for the fluid transfer system, and data stored in the second transponder contain identification data for the tubular line.

3. The connecting system of claim 1, wherein at least one of the first transponder and the second transponder is an RFID transponder.

4. The connecting system of claim 1, wherein at least one of the first transponder and the second transponder has a read-only memory (ROM) with an identification code unchangeably stored therein as well as both a readable and writable second memory.

5. The connecting system of claim 1, wherein the plug-in component is disposed on one end of the tubular line, and the other end of the tubular line comprises a connector for connecting a cannula.

6. The connecting system of claim 1, wherein the fluid transfer system is a cassette insertable into an injection system and which comprises a plurality of input ports for delivery of medical fluids and a plurality of fluid channels and valves, and the output port of the cassette is formed by the coupling component.

7. An injection system for injection of a medical fluid, comprising a housing, a delivery system for delivery of the fluid, a fluid transfer system, which is connectable to at least one fluid container and which exchangeably inserted in the housing, and a tubular line which is connected to the fluid transfer system, wherein the fluid transfer system is connected to the tubular line via the connecting system of claim 1.

8. The injection system of claim 7, wherein a read/write unit is disposed on the housing of the injection system.

9. The injection system of claim 7, wherein data stored in the first transponder contain an identification code for identification of the fluid transfer system, which code is stored in a read-only memory of the first transponder, and data stored in the second transponder contain an identification code for identification of the tubular line, which code is stored in a read-only memory of the second transponder.

10. A method of operating the injection system of claim 7, the method comprising:
a. inserting the fluid transfer system into the housing of the injection system and connecting the fluid transfer system to at least one fluid container,
b. establishing a connection between the fluid transfer system and the tubular line via the connecting system,
c. reading the identification data for the fluid transfer system stored in the first transponder and the identification data for the tubular line stored in the second transponder via the read/write unit.

11. The method of claim 10, wherein prior to starting delivery of a fluid to be injected, data are read from the first transponder and/or from the second transponder, and the data read are compared to reference data, with delivery of the fluid to be injected being started only if the data read conform at least within a predefined range to the reference data.

12. The method of claim 10, wherein based on the data read from the memory of the first transponder and/or of the second transponder prior to starting delivery of the fluid to be injected, it is determined whether and, optionally, at what point in time a fluid to be injected has previously been delivered for the first time by the tubular line and/or the fluid transfer system, and delivery at the current point in time is started only if the difference in time between the current point in time and the point in time of the first use read is below a predefined threshold limit

13. The method of claim 12, wherein the predefined threshold limit of the difference in time for the tubular line differs from that for the fluid transfer system, with the threshold limit for the tubular line preferably being lower than the threshold limit for the fluid transfer system.

14. The method of claim 12, wherein the point in time of a first use of the tubular line is written to a memory of the first transponder if, based on the data read from the first transponder prior to starting delivery of a fluid to be injected, it is determined that the tubular line will be used for the first time, and/or that the point in time of the first use of the fluid transfer system is written to a memory of the second transponder if, based on the data read from the second transponder prior to starting delivery of the fluid to be injected, it is determined that the fluid transfer system will be used for the first time.

15. The method of claim 10, wherein the length of time of use to date and/or the allowed remaining length of time of use of the fluid transfer system and/or the tubular line connected thereto is displayed on a display unit of the injection system, and/or that an error message is displayed on a display unit of the injection system if the data read from the first and/or the second transponder are outside of a predefined range of allowable values or do not conform to the reference values.
